(19) United States
(12) Patent Application Publication
(10) Pub. No.: US 2008/0245365 A1
(43) Pub. Date: Oct. 9, 2008

(54) Evaporators and Evaporation Methods

(75) Inventors: Harald Genger, Dessau (DE); Martin Baecke, Dessau (DE); Ulla Schoebel, Koethen (DE)

Correspondence Address: AKC PATENTS, 215 GROVE ST., NEWTON, MA, 02466, US

(73) Assignee: SELEON GMBH, DESSAU (DE)

(21) Appl. No.: 11/573,058

(22) PCT Filed: Aug. 1, 2005
(86) PCT No.: PCT/DE05/01351

§ 371 Date: Aug. 7, 2007

(30) Foreign Application Priority Data
Aug. 2, 2004 (DE) 10 2004 037 698.0

(51) Int. Cl. A61M 16/16; F24F 6/10; B01D 47/02

(52) U.S. Cl. 128/204.14; 392/394; 392/400; 128/203.12; 261/74

(57) ABSTRACT

This invention relates to an evaporator comprising a casing (1; 21) and a heater (7; 27). Gas is supplied through an inlet (4; 24) and discharged through an outlet (5; 25). The casing forms an evaporating space (2; 22) and a heating space (3; 33) heated by the heater (7; 27). The evaporating space (2; 22) may be partially filled with a liquid (16; 36). The gas flows from the inlet (4; 24) via the heating space (3; 23) to the evaporating space (2; 22) and further to the outlet (5; 25). This invention moreover relates to an evaporator comprising an outer and an inner tub. The outer tub is heated. The inner tub may contain a water reserve and is inserted into the outer tub. The upper side of the bottom of the outer tub (8; 28) or the underside of the bottom of the inner tub (10; 30) is made of a soft elastic material. The invention finally relates to corresponding methods.

### CROSS REFERENCE TO RELATED CO-PENDING APPLICATIONS

This application is an entry into national stage under PCT of international application number PCT/DE2005/001351 (publication number: WO 2006/012877 A1) filed on Aug. 1, 2005 and entitled EVAPORATORS AND EVAPORATION METHODS and claims the benefit of the above-mentioned international application and the corresponding German national patent application number 10 2004 037 698.0 filed on Aug. 2, 2004 and entitled VERDUNSTER SOWIE VERDUNSTUNGSVERFAHREN the contents of which are expressly incorporated herein by reference.

### FIELD OF THE INVENTION

The invention relates to an evaporator of the type defined in the preambles of patent claims 1 and 19 as well as to methods of the type defined in the preambles of patent claims 13 and 21. More specifically, the invention relates to humidifiers for respirators in a broader sense, i.e. also to the field of CPAP-, bi-level and anti-snore apparatus.

### BACKGROUND OF THE INVENTION

Respirators include so-called CPAP-apparatus which serve the treatment of apneas during the sleep. To this end, the CPAP (continuous positive airway pressure) therapy was developed. A CPAP-apparatus generates a positive airway pressure up to approximately 30 mbar by means of a compressor or turbine and administers the same, preferably via a humidifier, via a tube and via a nose mask, to the respiratory tract of the patient. This positive airway pressure is to ensure that the upper respiratory tract remains fully opened during the whole night, so that no apneas will occur (DE 198 49 571 A1). This is also referred to as pneumatic splinting of the respiratory tract. A humidifier used in conjunction with said CPAP-apparatus prevents the patient's mucous membranes from desiccating.

An anti-snore apparatus is known from DE 101 05 383 C2, wherein air spectacles are used instead of a nose or face mask. An advantage of this anti-snore apparatus is the better wear property resulting from the avoidance of a nose or face mask and from the use of thinner tubes. Also, air spectacles are similar to oxygen spectacles, which are already produced in large numbers of pieces at low costs.

A respiratory gas humidifier for CPAP-apparatus is described in DE 199 36 499 A1. The humidifier comprises a refill unit formed of a tub element and a pot part coupled therewith, which can be removed from a mountable casing. The tub element and the pot part are imperviously connected with each other. In conjunction with a partition wall a store room for a liquid is formed in said pot part, which contains the major part of the water reserve provided for humidifying the respiratory gas. A separate humidifying space is formed in the tub element disposed underneath the pot part, which merely contains a small portion of the water reserve. The height of the water in the tub element is kept at a predetermined level by a dosing device. In the course of the gradual evaporation of the water located in the tub element water from the liquid store room is successively or continuously refilled. Via a respiratory gas inlet opening the respiratory gas is blown through the upper portion of the tub element to a respiratory gas outlet opening. The bottom area of the tub element is heated by a heating device.

A humidifier for respirators similar to the one described in DE 199 36 499 A1 is described in DE 200 10 553 U1. In the humidifier according to DE 200 10 553 U1 the air is also passed over the surface of a heatable water reservoir. A water tank, which is substantially integral, is used instead of the refill unit formed of a tub element and a pot part.

Other similar humidifiers are known from DE 101 51 397 C1 and the German Utility Model 20 2004 004 115.4.

It is desirable to provide an evaporator and an evaporation method capable of providing a fast operational availability.

### SUMMARY OF THE INVENTION

According to an embodiment of the invention an evaporator comprises a heater and a casing. The casing has an inlet for supplying a gas and an outlet for discharging the supplied gas. The casing forms an evaporating space which may partially be filled with a liquid and a heating space which is in thermal contact with the heater. The inlet is pneumatically connected to the heating space. The heating space is pneumatically connected to the evaporating space. The evaporating space is pneumatically connected to the outlet. Consequently gas can flow from the inlet through the heating space to the evaporating space and further to the outlet. The casing comprises an inner tub which is open at the top and closed at the bottom for receiving the liquid. The inner tub separates the evaporating space from the heating space.

According to another embodiment of the invention an evaporation method is provided. A gas is heated in a heating space positioned between an inner tub and an outer tub. The outer tub is heated. The gas is conducted over a liquid surface after the gas has been heated in the heating space and the gas is conducted in an evaporating space over the liquid surface. The evaporating space and the liquid being located inside the inner tub.

According to a further embodiment of the invention an evaporator is provided which comprises an outer and inner tub and a heater. The heater is thermally coupled to the outer tub; The inner tub may be inserted into the outer tub and may receive a water reserve. The upper side of the bottom of the outer tub or the underside of the bottom of the inner tub is made of a soft elastic material.

According to yet a further embodiment of the invention a evaporation method is provided. The method comprises filling an inner tub with a liquid. The inner tub is inserted into an outer tub. The outer tub is heated. The underside of the bottom of the inner tub is adapted to the upper side of the bottom of the outer tub or the upper side of the bottom of the outer tub is adapted to the underside of the bottom of the inner tub.

Due to the small thermal capacity of the supplied gas, as compared to the thermal capacity of the liquid reserve, a significant evaporation is achieved virtually immediately after the initial operation.

By heating the inflowing gas the entire liquid surface, including the inlet space, is used for the evaporation of liquid molecules or atoms. If, on the other hand, as provided in conventional humidifiers, the liquid is heated, the gas in the inlet space is heated to the temperature of the liquid so that the inlet space is not effectively used for the evaporation because cooler gas absorbs liquid molecules or atoms less readily.

A removable inner tub, which serves as a storage tank for the liquid, facilitates the refilling of liquid and the cleaning of the evaporator.

Pressure tightness provides that an evaporator acts like a piece of pipe for the gas.

By uniformly heating the outer tub the required heating power is conducted through a large surface. This results in a lower temperature at the heater.

An advantage for an inexpensive production is that no sealing is required between the inner tub and the lid, but that there may even be a narrow gap. The different gas resistance between the narrow gap and the aperture is rather sufficient to allow the major part of the gas to flow through the aperture.

Moreover, the inventive embodiments require no moving sealing at the inlet and the outlet, which keeps the production costs low and increases reliability.

The thermal transport from the heated outer tub toward the inner tub by means of the liquid is supported by the movement of the gas between the outer and inner tub, namely possibly in the entire gap. By a lateral transport of material in a turbulent gas flow the thermal conductivity of moving gases is higher than that of static gases. Additionally, heat is transported by the gas flow through the aperture and transferred from the bottom of the outer tub to the bottom of the inner tub. All this provides for a good thermal coupling between the heater, the gas and the liquid, so that lower heater temperatures are sufficient to compensate an evaporation cold. Lower heater temperatures also reduce problems with respect to calcification.

By arranging the aperture backward of the inlet, seen in the direction of circulation of the gas, it is achieved that the gas moves over the entire length of the gap.

A high relative speed between the liquid and the gas favors an evaporation of liquid molecules or atoms.

An inner tub having two cylindrical portions and an intermediate waisted portion fits well into an oval outer tub. The gap between the inner and the outer tub may be narrow in the cylindrical portions, which provides for a good heat transfer due to the small distance between the inner and the outer tub and the high flow rate of the gas and the turbulences resulting therefrom. The distance between the inner and the outer tub in the waisted portion is larger, so that the gap is here wide enough for a normally sized finger to allow the easy removal of the inner tub for cleaning or refilling water.

The two tubs and the lid may advantageously be made of stainless steel. As compared with plastics, stainless steel is able to withstand high temperatures and is additionally biocompatible. Especially fire prevention additives for plastics are mostly not biocompatible.

### BRIEF DESCRIPTION OF THE DRAWINGS

Preferred embodiments of the invention are explained in more detail below, with reference to the attached drawings, wherein like numerals represent like parts. In the drawings:

FIG. 1 shows a top view of an evaporator according to the invention;

FIG. 2 shows a lateral view of the evaporator illustrated in FIG. 1;

FIG. 3 shows a top view of another evaporator according to the invention;

FIG. 4 shows a lateral view of the evaporator illustrated in FIG. 3;

FIGS. 5 to 7 show vertical sections through different embodiments of the inner tub according to the invention.

### DETAILED DESCRIPTION OF THE INVENTION

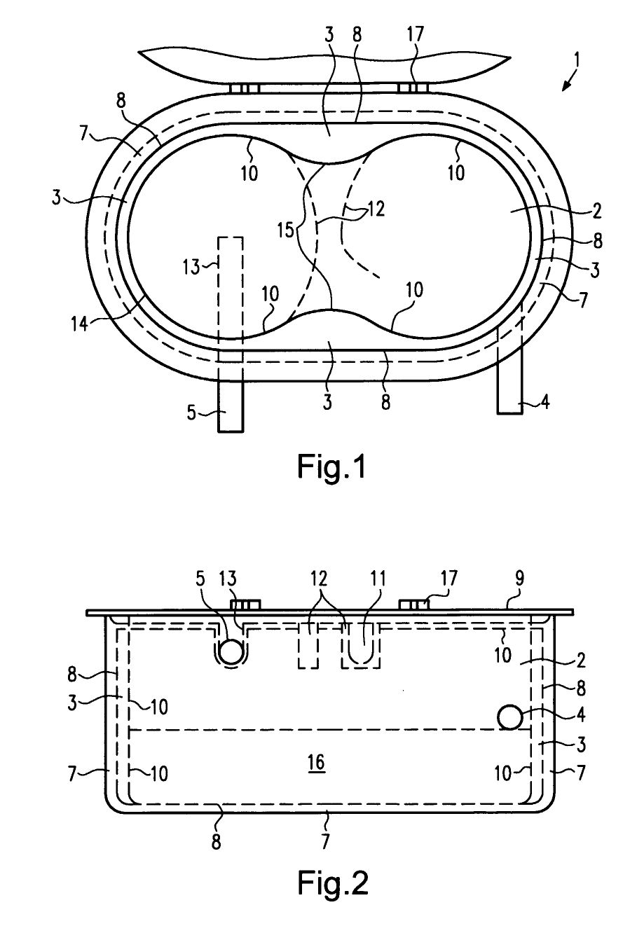

FIGS. 1 and 2 show an embodiment of the evaporator according to the invention. The casing 1 of the evaporator is substantially formed of three parts, namely an outer tub 8, an inner tub 10 and a lid 9. The inner tub 10 stands in the outer tub 8 so that the two tub bottoms are in contact with each other and heat can also be transferred over the bottoms. In FIG. 1, only a part of the lid 9 is shown, namely in an opened position. The lid 9 may be attached to the outer tub 8 by means of hinges 17.

Between the circumferential surfaces of both tubs a gap is formed, which is referred to as heating space 3. The space enclosed by the inner tub 10 and the lid 9 is referred to as evaporating space 2. In the lower part of the evaporating space a liquid is located, mostly water 16. Outside the outer tub 8, i.e. both at the bottom surface and the circumferential surface, a heater 7 is provided.

Gas, mostly air, is supplied to the evaporator through the inlet 4. The gas first flows into the heating space 3, where it flows counterclockwise around the inner tub 10 nearly once. By this the gas on the circumferential surface of the outer tub 8 is heated and transfers a part of the heat also to the inner tub 10. The gas then flows through the aperture 11 from the heating space 3 into the evaporating space 2. By internal parts 12 in the lid 9, the position of which with lid 9 being closed is shown as dashed lines in FIG. 1, it is achieved that the gas is passed possibly over the entire liquid surface. This favors the evaporation of the liquid. Finally, the gas is discharged through the outlet channel 13 and the outlet 5.

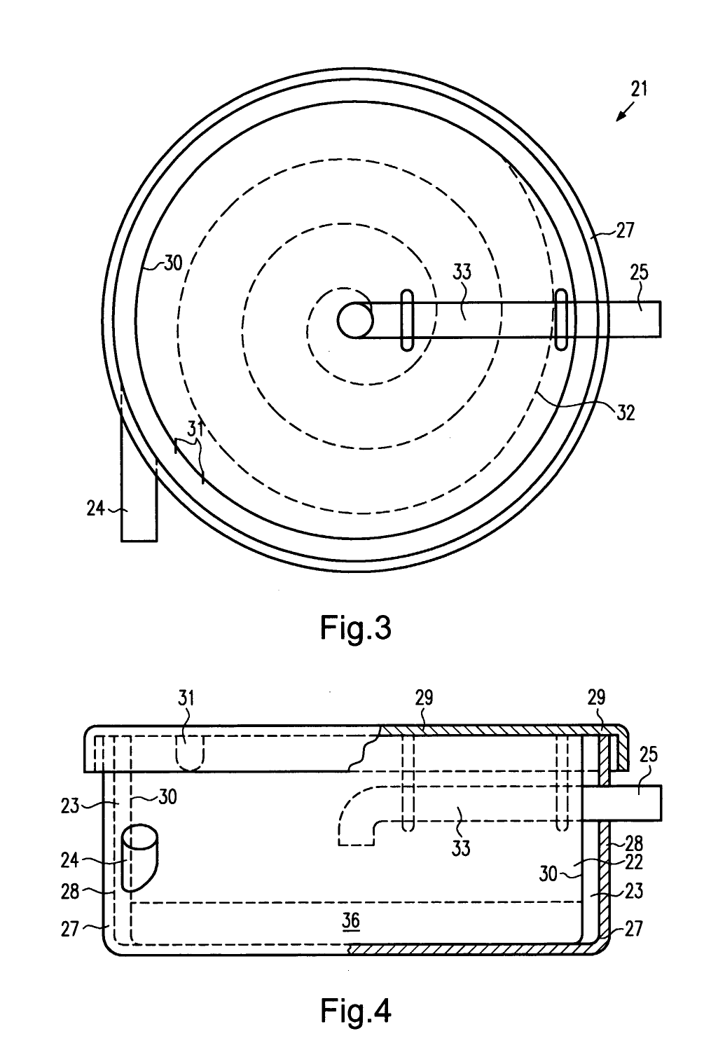

Another embodiment may be set up mirror-symmetrically to the embodiment shown in FIGS. 1 and 2, so that the gas flows clockwise around the inner tub 10, such as in the embodiment shown in FIGS. 3 and 4.

The only high-grade and moving sealing is located between the outer tub 8 and the lid 9. The gap seen in FIG. 2 between the inner tub 10 and the lid 9 should be as narrow as possible to allow the gas to flow through the aperture 11 in a defined manner. On the other hand, the functions of the evaporator are not substantially affected if, for example, one half of the gas flows through the gap while the other half flows through the aperture 11.

As can well be seen in FIG. 1 the inner tub 10 has a bone-shaped outline with two cylindrical portions 14 and an intermediate waisted portion 15. The purpose of the waisted portion 15 resides above all in providing space for normally sized fingers in the waisted portion 15 between the inner and the outer tub. This, again, ensures that the inner tub can be removed conveniently from the outer tub for refilling or cleaning purposes.

The waisted portion 15 also allows the gas flow in the heating space 3 to become turbulent. Therefore, the heat transfer from the circumferential surface of the outer tub via the gas in the heating space 3 onto the circumferential surface of the inner tub will not deteriorate to an extent that would be assumed because of the larger distance between the two circumferential surfaces in the waisted portion.

To allow a sweeping of the gas possibly over the entire liquid surface the aperture 11 is located in the one cylindrical portion and the outlet channel 13 is located in the other cylindrical portion.

FIGS. 3 and 4 show another embodiment of the evaporator according to the invention. In contrast to the evaporator shown in FIGS. 1 and 2, the casing 21 of this evaporator including the outer tub 28, the inner tub 30 and the lid 29 are substantially rotationally symmetric. Here, too, the gas is blown through an inlet 24 into an annular heating space 23 between the circumferential surfaces of the inner and the outer tub. After the gas has flown nearly once around the inner tub 30 it flows through the aperture 31 into the evaporating space 22 inside the inner tub 30, where it sweeps over the surface of a liquid, e.g. water 36. The gas, humidified or accumulated with liquid molecules or atoms, is then conducted out of the center of both tubs through the outlet channel 33 toward the outlet 25. This embodiment, too, can be mirrored.

The lid 29 may comprise spiral-shaped internal parts 32 shown as dashed lines in FIG. 3, which allow the use of the entire liquid surface for the evaporation.

As shown in FIGS. 3 and 4, sharp corners especially on the tubs, but also on the lids are avoided so as to facilitate the cleaning of the parts. Especially, the edges between the circumferential surfaces and the bottoms of the tubs are rounded. This cleaning friendliness allows the permanent installation of the evaporator in a larger apparatus to avoid by this moving sealings at the inlet and the outlet.

The outer and inner tubs as well as the lid may be made of stainless steel. Stainless steel is biocompatible and withstands high temperatures, while fire prevention agents in plastics are mostly not biocompatible.

The heater 7 may be made, for example, of a wound heating wire fixed and insulated with adhesive tape, or of a Kapton film heating.

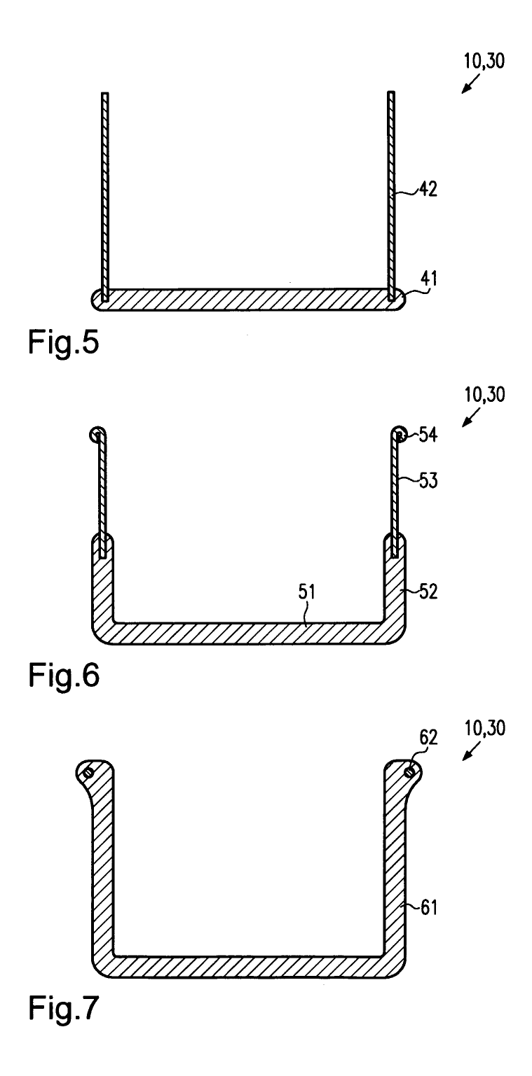

The inner tub only schematically shown in FIG. 1 to 4 may also be realized as illustrated in one of FIG. 5 to 7. FIG. 5 to 7 show vertical sections through inner tubs 10 or 30.

In the embodiments shown in FIG. 5 to 7 the bottom of the inner tub is made of an elastic biocompatible material, e.g. of silicone. This has the advantage that the bottom of the inner tub adapts to the bottom of the outer tub and that an air gap between the inner and the outer tub is decreased and, in the ideal case, is reduced to zero over the entire bottom surface. This reduces the thermal resistance between the two bottoms and further reduces the temperature of the heater.

Although the bottom of the inner tub should be soft to allow its adaptation to the bottom of the outer tub, the walls of the inner tub must have a certain rigidity to enable the grasping thereof when removing it from the outer tub.

The bottom of the outer tub, too, can be made a soft elastic material such as silicone, or may be coated with such a material. Such an outer tub may be combined with an inner tub which is made of stainless steel or with an inner tub the bottom of which is made of silicone.

FIG. 5 shows an embodiment of the inner tub the bottom 41 of which is made of silicone, while the walls 42 are made of stainless steel.

FIG. 6 shows an embodiment of the inner tub the bottom 51 of which and the lower portion of the circumferential surface 52 are made of silicone, while the upper portion of the circumferential surface 53 is made of stainless steel. The upper edge 54 of the circumferential surface may be bent over.

In the embodiment shown in FIG. 7 the entire inner tub is made of silicone 61. The upper edge of the tub is broadened so as to increase the stability. To further increase the stability a wire 62, especially a spring wire, may be embedded in the upper edge.

Due to heating the gas a significant evaporation of the liquid is achieved virtually immediately after the initial operation. After the initial operation the evaporation rate increases until the liquid has reached its equilibrium temperature. To accelerate this process a preheating for three to four minutes before the initial operation may be advantageous.

The evaporators according to the invention are primarily used as humidifiers. However, this is not a necessary feature because it is known from the German patent application 103 22 964.7 to use a gas mixture having a composition which deviates from air or to add substances such as aerosols, aromas or medicine to the air. A substance such as a sea water additive may also be added to the water, so that the generic term “liquid” is justified.

### LIST OF REFERENCE NUMERALS

1 casing
2 evaporating space
3 heating space
4 inlet
5 outlet
7 heater
8 outer tub
9 lid
10 inner tub
11 aperture
12 internal parts
13 outlet channel
14 cylindrical portions
15 waisted portion
16 water
17 hinge
21 casing
22 evaporating space
23 heating space
24 inlet
25 outlet
27 heater
28 outer tub
29 lid
30 inner tub
31 aperture
32 internal parts
33 outlet channel
36 water
41 bottom
42 circumferential surface
51 bottom
52 lower circumferential surface
53 upper circumferential surface
54 bent-over edge
61 silicone
62 wire

Several embodiments of the present invention have been described. Nevertheless, it will be understood that various modifications may be made without departing from the spirit and scope of the invention. Accordingly, other embodiments are within the scope of the following claims.

What is claimed is:

1-21. (canceled)

22: An evaporator, comprising:
a heater;
a casing having an inlet for supplying a gas and an outlet for discharging the supplied gas, wherein the casing forms an evaporating space which is adapted to be partially filled with a liquid;
the casing forming a heating space being in thermal contact with the heater, the inlet being pneumatically connected to the heating space and the heating space being pneumatically connected to the evaporating space and the evaporating space being pneumatically connected to the outlet to allow gas to flow from the inlet through the heating space to the evaporating space and further to the outlet; and
the casing comprising an inner tub being open at the top and closed at the bottom for receiving the liquid, the inner tub separating the evaporating space from the heating space.

23: The evaporator according to claim 22, wherein the casing comprises an outer tub and a lid and wherein the outer tub and the lid are closable in a pressure-tight manner.

24: The evaporator according to claim 23, wherein the heater is attached outside on the outer tub so that it heats the outer tub largely uniformly.

25: The evaporator according to claim 22, wherein the casing comprises an outer tub, and a lid, said outer tub and said lid being closable in a pressure-tight manner, wherein the inner tub stands in the outer tub and the upper edge of the inner tub reaches as far as the lid, wherein the lid and the inner tub enclose the evaporating space and wherein the heating space is located between the inner and the outer tub and is additionally limited by the lid.

26: The evaporator according to claim 25, wherein the inlet is formed such that gas flowing into the heating space flows into the same tangentially, i.e. approximately parallel to the walls of the inner and the outer tub so that the design of the inlet defines a direction of circulation of the gas in the heating space around the inner tub.

27: The evaporator according to claim 26, wherein an aperture is located in the inner tub, wherein the aperture connects the heating space to the evaporating space and wherein the aperture is located just backward of the inlet seen in the direction of circulation.

28: The evaporator according to claim 23, wherein the lid is provided with internal parts for conducting the gas, which provide for a high relative speed between the liquid and the supplied gas during operation.

29: The evaporator according to claim 27, wherein the circumferential surface of the inner tub has two cylindrical portions connected by a waisted portion, wherein the aperture is located in the one cylindrical portion and an outlet channel extends from the center of the other cylindrical portion toward the outlet.

30: The evaporator according to claim 27, wherein the circumferential surfaces of the inner and outer tub are cylindrical and an outlet channel extends from the center of the inner tub to the outlet.

31: The evaporator according to claim 23, wherein one of the inner tub, the outer tub and the lid are made of stainless steel.

32: The evaporator according to claim 23, wherein the outer tub is made of stainless steel and the bottom surface of the inner tub is made of silicone.

33: An evaporation method, comprising
heating a gas in a heating space positioned between an inner tub and an outer tub;
heating the outer tub;
conducting the gas over a liquid surface after the gas has been heated in the heating space; and
conducting the gas in an evaporating space over the liquid surface, the evaporating space and the liquid being located inside the inner tub.

34: The evaporation method according to claim 33, further comprising:
tangentially introducing the gas into the heating space so that the gas flows around the inner tub thereby defining a direction of circulation.

35: The evaporation method according to claim 34, further comprising:
transferring the gas from the heating space into the evaporating space at a location which, seen in the direction of circulation, is just backward of the location where the gas is introduced into the heating space.

36: The evaporation method according to claim 33, wherein the gas is conducted spirally into a cylindrical portion of the evaporating space toward the center of the cylindrical portion and from there through an outlet channel toward an outlet.

37: The evaporation method according to claim 33, further comprising:
inserting the inner tub into the outer tub; and
adapting the shape of the bottom of the inner tub to the shape of the bottom of the outer tub with each insertion.

38: An evaporator, comprising:
an outer tub;
a heater thermally coupled to the outer tub;
an inner tub adapted to be inserted into the outer tub and to receive a water reserve; and
wherein the upper-side of the bottom of the outer tub or the under-side of the bottom of the inner tub comprises a soft elastic material.

39: The evaporator according to claim 38, wherein the soft elastic material is silicone.

40: An evaporation method, comprising:
filling an inner tub with a liquid;
inserting the inner tub into an outer tub;
heating the outer tub; and
adapting the underside of the bottom of the inner tub to the upper side of the bottom of the outer tub or adapting the upper side of the bottom of the outer tub to the underside of the bottom of the inner tub.
